(19) United States
(12) Patent Application Publication
(10) Pub. No.: US 2021/0378575 A1
(43) Pub. Date: Dec. 9, 2021

(54) Coated Electroencephalogram Electrodes

(71) Applicant: Dignity Health, San Francisco, CA (US)

(72) Inventors: Asim HAQUE, Grand Rapids, MI (US); Sarah BARAN, Chandler, AZ (US)

(21) Appl. No.: 17/342,090

(22) Filed: Jun. 8, 2021

Related U.S. Application Data
(60) Provisional application No. 63/036,532, filed on Jun. 9, 2020.

(51) Int. Cl. A61B 5/293; A61B 5/262

(52) U.S. Cl. CPC A61B 5/293; A61B 2562/0217; A61B 5/262

(57) ABSTRACT

Subdermal electroencephalogram (EEG) electrodes are coated to minimize pain and bleeding, and to improve electrical signal transmission.

The present application claims the benefit of the filing date of U.S. provisional application Ser. No. 63/036,532, filed 9 Jun. 2020.

### FIELD OF THE INVENTION

The invention generally relates to coating subdermal electroencephalogram (EEG) electrodes to minimize pain and bleeding.

### BACKGROUND

The EEG provides the medical practitioner with important information concerning brain activity. As disclosed in U.S. Pat. No. 9,345,418, there are improved systems and methods for the implementation of mobile techniques in obtaining EEG information in an urgent care setting. Without requiring a high level of training, these techniques allow technician to quickly apply EEG electrodes to a patient in the field. Commonly, placement of the electrodes conforms with the International 10-20 System. Typically, each electrode is electrically connected to one input of a differential amplifier, and a common system reference is electrically connected to the other input of each differential amplifier. The voltage difference between the active input and the reference input is amplified, filtered, and converted to a digital signal. These signals are representative of electrical brain activity of the patient. The EEG data can be transmitted in real time to a doctor at a remote location, who is assessing the patient's situation and communicating instructions.

The EEG data is often of greater quality and reliability if subdermal electrodes are used. For example, needle electrodes or pronged electrodes may be used in which a needle point or prong points protrude from an electrode head and are adapted to be embedded in a patient's scalp. While the quality of the data may be better, use of subdermal electrodes can cause pain and bleeding.

### BRIEF DESCRIPTION OF THE DRAWINGS

The following drawings illustrate the concepts of the present invention. Illustrations of an exemplary device are not necessarily drawn to scale.

FIG. 1 is a perspective view of a generic pronged electrode.

FIG. 2 is a perspective view of a generic needle electrode.

FIG. 3 depicts a frontal view of a support structure mounted on a patient's head, with a plurality of electrodes mounted on the support structure.

### DETAILED DESCRIPTION

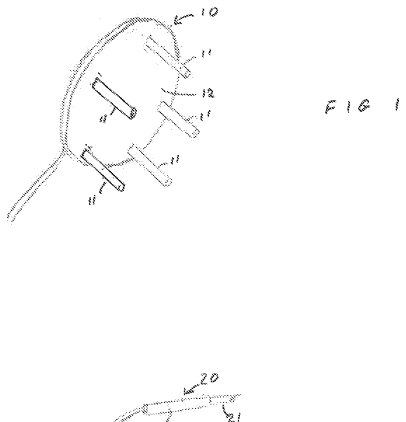

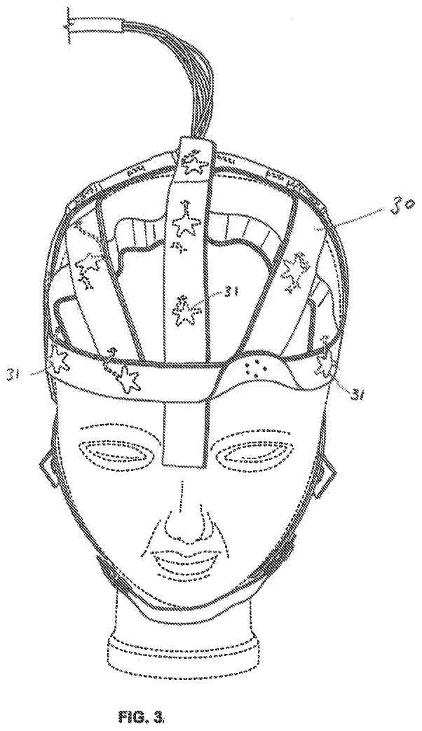

FIGS. 1 and 2 are illustrations of generic pronged and needle electrodes, respectively. These are examples of subdermal EEG electrodes which are intended to be embedded in a patient's scalp. Electrode 10 with prong points 11 extending from an electrode head 12 is representative of a pronged electrode, and electrode 20 with needle point 21 extending from an electrode head 22 is representative of a needle electrode.

In an embodiment, the points 11 or 21 are coated with a local anesthetic to minimize pain associated with embedding those electrode points in a patient's scalp. An example of such an anesthetic is lidocaine.

In an embodiment, the points 11 or 21 are coated with a coagulant to minimize bleeding associated with embedding those electrode points in a patient's scalp. An example of such a coagulant is epinephrine. Combining both lidocaine and epinephrine in the coating protects against pain and bleeding. Another advantage of doing so is that epinephrine is an antidote for the allergic reaction that a very small population would experience with the application of lidocaine.

In an embodiment, the points 11 or 21 are coated with a conductive gel to improve the electrical conductivity between the patient and the electrode points 11 or 21, The conductive gel can be combined with the anesthetic, or the coagulant, or both in the coating.

The electrode points 11 or 21 can be pre-coated during manufacture. Alternatively, the coating can be provided in a separate, sealed container, and applied to the electrode points 11 or 21 just before the electrode 10 or 20 is applied to a patient's head.

FIG. 3 depicts an example of a support structure 30 mounted on a patient's head. In some embodiments, the support structure 30 may be an elastic net or cap. A plurality of EEG electrodes 31 are mounted on the support structure 30. The support structure 30 is adapted to cover at least predetermined areas on a patient's head, so that when the electrodes 31 are mounted on the support structure 30 and the support structure 30 is mounted on a patient's head, the electrodes are distributed around the patient's head and are capable of detecting electrical signals that are representative of the patient's electrical brain activity. The electrodes are subdermal electrodes that are coated as described above.

It will be understood that the disclosed embodiments can be modified without departing from the teachings of the invention. Accordingly, the scope of the invention is only to be limited as necessitated by the accompanying claims.

What is claimed is:

1. A subdermal electroencephalogram (EEG) electrode, comprising:
an electrode head; and
at least one point extending from the head and adapted to be embedded in a patient's scalp;
wherein the at least one point is coated with a coagulant.

2. The subdermal EEG electrode of claim 1, wherein the at least one point is coated with a conductive gel.

3. The subdermal EEG electrode of claim 1, wherein the coagulant comprises epinephrine.

4. The subdermal EEG electrode of claim 1, wherein the at least one point is coated with a local anesthetic.

5. The subdermal EEG electrode of claim 4, wherein the local anesthetic comprises lidocaine.

6. The subdermal EEG electrode of claim 4, wherein the at least one point is coated with a conductive gel.

7. An EEG system, comprising:
a support structure that is adapted to cover at least predetermined areas on the patient's head; and
a plurality of electrodes, each of the electrodes being a subdermal EEG electrode according to claim 1;
wherein, when the plurality of electrodes are mounted on the support structure and the support structure is mounted on the patient's head, the electrodes are distributed around the patient's head and are capable of detecting electrical signals that are representative of the patient's electrical brain activity.

8. The EEG system of claim 7, wherein the at least one point of each of the electrodes is coated with a conductive gel.

9. The EEG system of claim 7, wherein the coagulant comprises epinephrine.

10. The EEG system of claim 7, wherein the at least one point of each of the electrodes is coated with a local anesthetic.

11. The EEG system of claim 10, wherein the local anesthetic comprises lidocaine.

12. The EEG system of claim 10, wherein the at least one point of each of the electrodes is coated with a conductive gel.

13. A method of preparing a subdermal electroencephalogram (EEG) electrode for insertion into a patient's scalp, the method comprising:
coating at least a portion of the electrode with a coating comprising a coagulant, in order to minimize bleeding associated with inserting the electrode into the patient's scalp.

14. The method of claim 13, wherein the coating further comprises a conductive gel, in order to improve electrical conductivity between the patient and the electrode.

15. The method of claim 13, wherein the coagulant comprises epinephrine.

16. The method of claim 13, wherein the coating further comprises a local anesthetic, in order to minimize pain associated with inserting the electrode into the patient's scalp.

17. The method of claim 16, wherein the local anesthetic comprises lidocaine.

18. The method of claim 6, wherein the coating further comprises a conductive gel, in order to improve electrical conductivity between the patient and the electrode.
